(19) United States
(12) Patent Application Publication
(10) Pub. No.: US 2023/0346226 A1
(43) Pub. Date: Nov. 2, 2023

(54) PHANTOM FOR AN OPTOACOUSTIC PROBE AND METHOD OF MAKING THE SAME

(71) Applicant: Seno Medical Instruments, Inc., San Antonio, TX (US)

(72) Inventors: Tam Do, San Antonio, TX (US); Jason Cook, San Antonio, TX (US); Lisa Michelle Richards Cook, San Antonio, TX (US)

(73) Assignee: Seno Medical Instruments, Inc., San Antonio, TX (US)

(21) Appl. No.: 18/303,859

(22) Filed: Apr. 20, 2023

Related U.S. Application Data
(60) Provisional application No. 63/363,763, filed on Apr. 28, 2022.

(51) Int. Cl. A61B 5/00

(52) U.S. Cl. CPC A61B 5/0095; A61B 5/4312; A61B 8/587

(57) ABSTRACT

A method for forming a phantom for an optoacoustic probe is provided that may include forming a scattering material. The method may also include forming an absorbing material, and mixing the scattering material with the absorbing material to form a tissue material. The method can also include mixing the tissue material with a urethane solution to form a phantom material, and curing the phantom material to form the phantom.

### CROSS REFERENCE TO RELATED APPLICATIONS

This application claims benefit to U.S. Provisional Application No. 63/363,763, filed on Apr. 28, 2022, titled “PHANTOM FOR AN OPTOACOUSTIC PROBE AND METHOD OF MAKING THE SAME”, the complete subject matter of which is expressly incorporated herein by reference in its entirety.

### COPYRIGHT PROTECTIONS

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### TECHNICAL FIELD

The present invention relates in general to the field of medical imaging, and in particular to a phantom for an optoacoustic probe and a method of manufacturing the phantom.

### BACKGROUND

Optoacoustic imaging systems visualize thin features noninvasively through skin at a tissue site. A tissue site may contain a variety of tissue structures that may include, for example, tumors, blood vessels, tissue layers, and components of blood. In optoacoustic imaging systems, light is used to deliver optical energy to a volume of the tissue site, which as a result of optical absorption with the tissue structures, produce acoustic (and more generally pressure) waves. An image spatially representing information from the tissue site can be generated by performing image reconstruction on acoustic signals that return to an ultrasound transducer array. Because biological tissue alters optical energy in many directions the optical energy can be absorbed by tissue structures outside of a targeted region, which can generate acoustic return signals that interferes with the imaging of tissue structures within the targeted region.

The proper use of an optoacoustic imaging system includes training and practice identifying irregularities including masses, tumors, other irregularities, or the like utilizing an optoacoustic probe. One way of training individuals, doctors, practitioners, etc. is to utilize a phantom. A phantom is mimic of the pertinent properties of a body part that is typically imaged with an optoacoustic imaging system. In one example, the phantom may replicate, or mimic, a breast, and includes numerous targets, or irregularities within the phantom that can aid training of proper system usage. By having the individual train using the phantom, the individual becomes comfortable with the optoacoustic probe, improving the likelihood that an irregularity will be detected.

Additionally, the ability to perform quality assurance checks on the performance of an optoacoustic imaging system is important. One way of performing quality assurance checks is by utilizing a phantom that replicates the acoustic and optical properties of the tissue site relevant to optoacoustic imaging with known target geometries which can be measured and compared periodically. In one example, the phantom may mimic the optical and acoustic properties of a breast with target geometries that the individual must locate and measure.

When manufacturing a phantom, great detail must be undertaken to replicate the properties of the body part, as the acoustic and optical can affect the reading of the optoacoustic probe. In addition, the texture and consistency of the phantom material must also closely replicate the body tissue that will be examined in real life. Still, phantoms are often formed from materials that degrade easily and discolor over time. For example, gel wax phantoms can be prone to degradation, changes in color, and the like. In addition, gel wax phantoms do not match tissue properties as desired. Meanwhile, longer lasting phantoms, such as urethane based phantoms can have difficulties in absorbing ink, or other materials to facilitate coloring and placement of irregularities. This is especially true as compared to gel wax phantoms and other plastisol based phantoms. The advantage of a phantom that maintains its material properties over time is that it can be periodically measured in order to provide periodic quality assurance checks.

### BRIEF SUMMARY

New and useful systems, apparatuses, and methods for providing optoacoustic imaging are set forth in the appended claims. Illustrative embodiments are also provided to enable a person skilled in the art to make use of the claimed subject matter.

Objectives, advantages, and a preferred mode of making and using the claimed subject matter may be understood best by reference to the accompanying drawings in conjunction with the following detailed description of illustrative embodiments.

In accordance with embodiments herein, a method for forming a phantom for an optoacoustic probe is provided that may include forming a scattering material. The method may also include forming an absorbing material and mixing the scattering material with the absorbing material to form a tissue material. The method can also include mixing the tissue material with one or more urethane solutions to form a phantom material, and curing the phantom material to form the phantom.

Optionally, the scattering material includes TiO2 and a base material. In one aspect, the base material is mineral oil. In another aspect, the absorbing material includes carbon black with asphaltine and a base material. In one example, the method also includes determining whether the tissue material absorbs light related to a range of wavelengths. In another example, the range of wavelengths is at least one of between 740 nm and 770 nm, or 1050 nm-1070 nm. In yet another example, either one of additional scattering material or additional absorbing material is mixed with the tissue material based on determining whether the tissue material absorbs light related to the range of wavelengths.

Optionally, the curing of the phantom material to form the phantom occurs at a temperature in a range of between 65°-75° F. In one aspect, prior to mixing the tissue material with the urethane solution to form the phantom material includes sonification of tissue material. In another aspect, the method also includes placing the phantom material in a mold, and arranging one or more targets within the phantom material within the mold. In an example, the one or more targets include plural wire elements. In another example, the plural wire elements include a black wire element, red wire element, green wire element, and a clear wire element.

In one or more example embodiments, a method for forming a phantom for an optoacoustic probe is provided that includes forming a scattering material that includes TiO2 and a base material, and forming an absorbing material that includes carbon black with asphaltine and a base material. The method can also include mixing the scattering material with the absorbing material to form a tissue material, and mixing the tissue material with a urethane solution to form a phantom material. The method may also include curing the phantom material to form the phantom.

Optionally, the base material is mineral oil. In one aspect, the method can also include determining whether the tissue material absorbs light related to a range of wavelengths including at least one of between 740 nm and 770 nm, or 1050 nm-1070 nm. In another aspect, the curing of the phantom material to form the phantom occurs at a temperature in a range of between 65°-75° F. In one example, the method also includes placing the phantom material in a mold, and arranging one or more targets within the phantom material within the mold.

In one or more examples, a method for forming a phantom for an optoacoustic probe is provided that includes forming a scattering material that includes TiO2 and mineral oil, and forming an absorbing material that includes carbon black with asphaltine and mineral oil. The method also includes mixing the scattering material with the absorbing material to form a tissue material, and storing the tissue material. The method also includes performing sonication of the tissue material, and mixing the tissue material with a urethane solution to form a phantom material. The method also includes curing the phantom material to form the phantom.

Optionally, the method also includes determining whether the tissue material absorbs light related to a range of wavelengths. In one aspect, the range of wavelengths is at least one of between 740 nm and 770 nm, or 1050 nm-1070 nm.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a schematic block diagram illustrating an embodiment of an optoacoustic system that may be used as a platform for the methods and devices disclosed herein.

FIG. 2 shows a schematic block diagram illustrating a process for manufacturing a phantom for an embodiment disclosed herein.

FIG. 3 shows a schematic diagram of a phantom that may be utilized for the methods disclosed herein.

FIG. 4 shows a schematic block diagram of an example arrangement of manufacturing system used for the methods disclosed herein.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The systems and methods are described below with reference to, among other things, block diagrams, operational illustrations and algorithms of methods and devices to provide optoacoustic imaging with out-of-plane artifact suppression. It is understood that each block of the block diagrams, operational illustrations and algorithms and combinations of blocks in the block diagrams, operational illustrations and algorithms, can be implemented by means of analog or digital hardware and computer program instructions.

These computer program instructions can be stored on computer-readable media and provided to a processor of a general purpose computer, special purpose computer, ASIC, or other programmable data processing apparatus, such that the instructions, which execute via the processor of the computer or other programmable data processing apparatus, implements the functions/acts specified in the block diagrams, operational block or blocks and or algorithms.

In some cases, frequency domain-based algorithms require zero or symmetric padding for performance. This padding is not essential to describe the embodiment of the algorithm, so it is sometimes omitted from the description of the processing steps. In some cases, where padding is disclosed in the steps, the algorithm may still be carried out without the padding. In some cases, padding is essential, however, and cannot be removed without corrupting the data.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Reference will now be made in more detail to various embodiments of the present invention, examples of which are illustrated in the accompanying figures. As will be apparent to one of skill in the art, the data structures and processing steps described herein may be implemented in a variety of other ways without departing from the spirit of the disclosure and scope of the invention herein and should not be construed as being limited to the embodiments set forth herein. Rather, these embodiments are provided so that this disclosure will be thorough and complete, and will fully convey the concept of the disclosure to those skilled in the art.

Embodiments herein may be implemented in connection with one or more of the systems and methods described in one or more of the following patents, publications and/or published applications, all of which are expressly incorporated herein by reference in their entireties:

U.S. Pat. No. 7,999,161, titled “Laser-Activated Nanothermolysis Of Cells” filed Jul. 23, 2007;

U.S. Pat. No. 9,289,191, titled “System and method for Acquiring Optoacoustic Data and Producing Parametric Maps Thereof”, and filed Jun. 13, 2012;

U.S. Pat. No. 9,517,055, titled “System And Method For Acquiring Optoacoustic Data And Producing Parametric Maps Using Subband Acoustic Compensation” filed Nov. 25, 2013;

U.S. Pat. No. 9,724,072, titled “System And Method For Mixed Modality Acoustic Sampling” filed Dec. 13, 2013;

U.S. Pat. No. 9,456,805, titled “System And Method For Acquiring Optoacoustic Data And Producing Parametric Maps Using Interframe Persistent Artifact Removal” filed Dec. 19, 2013;

U.S. Publication 2016/0199037, titled “System And Method For Acquiring Optoacoustic Data And Producing Parametric Maps thereof” filed Mar. 22, 2016;

U.S. Publication 2017/0035388, titled “System And Method For Mixed Modality Acoustic Sampling” filed Oct. 18, 2016;

U.S. Pat. No. 9,792,686, titled “System And Method For Acquiring Optoacoustic Data And Producing Parametric Maps Using Subband Acoustic Compensation” filed Nov. 17, 2016;

U.S. Publication 2017/0296151, titled “System And Method For Mixed Modality Acoustic Sampling” filed Jun. 30, 2017;

U.S. Publication 2013/0109950, titled “Handheld Optoacoustic Probe” filed Nov. 02, 2011;

U.S. Publication 2016/0296121, titled “Handheld Optoacoustic Probe” filed May 02, 2016;

U.S. Pat. No. 8,686,335, titled “System And Method For Adjusting The Light Output Of An Optoacoustic Imaging System” filed Dec. 31, 2011;

U.S. Pat. No. 9,528,936, titled “System And Method For Adjusting The Light Output Of An Optoacoustic Imaging System” filed Mar. 31, 2014;

U.S. Publication 2017/0108429, titled “System And Method For Adjusting The Light Output Of An Optoacoustic Imaging System” filed Dec. 27, 2016;

U.S. Pat. No. 9,330,452, titled “Statistical Mapping In An Optoacoustic Imaging System” filed Mar. 11, 2013;

U.S. Pat. No. 9,836,838, titled “Statistical Mapping In An Optoacoustic Imaging System” filed May 03, 2016;

U.S. Publication 2018/0061050, titled “Statistical Mapping In An Optoacoustic Imaging System” filed Nov. 06, 2017;

U.S. Pat. No. 9,610,043, titled “System And Method For Producing Parametric Maps Of Optoacoustic Data” filed Jun. 13, 2012;

U.S. Publication 2017/0100040, titled “System And Method For Producing Parametric Maps Of Optoacoustic Data” filed Dec. 21, 2016;

U.S. Publication 2013/0338501, titled “System And Method For Storing Data Associated With The Operation Of A Dual Modality Optoacoustic/Ultrasound System” filed Jun. 13, 2012;

U.S. Publication 2013/0338475, titled “Optoacoustic Imaging System With Fiber Optic Cable” filed Jun. 13, 2012;

U.S. Publication 2014/0194723, titled “Multi-Layer Coating For Optoacoustic Probe” filed Jan. 13, 2014;

U.S. Publication 2017/0150890, titled “Optoacoustic Probe With Multi-Layer Coating” filed Jan. 31, 2017;

U.S. Pat. No. 9,615,750, titled “Methods And Compositions For Carrier Agents And Clearing Agents Used In Optoacoustic Imaging Systems” filed Jun. 14, 2012;

U.S. Publication 2013/0116538, titled “Optoacoustic Imaging Systems And Methods With Enhanced Safety” filed Oct. 19, 2012;

U.S. Publication 2015/0297090, titled “Optoacoustic Imaging Systems And Methods With Enhanced Safety” filed Jan. 23, 2015;

U.S. Publication 2013/0289381, titled “Dual Modality Imaging System For Coregistered Functional And Anatomical Mapping” filed Nov. 02, 2012;

U.S. Pat. No. 9,757,092, titled “Method For Dual Modality Optoacoustic Imaging” filed Nov. 02, 2012;

U.S. Publication 2014/0039293, titled “Optoacoustic Imaging System Having Handheld Probe Utilizing Optically Reflective Material” filed Jan. 22, 2013;

U.S. Publication 2017/0014101, titled “Dual Modality Imaging System For Coregistered Functional And Anatomical Mapping” filed Sep. 27, 2016;

U.S. Publication 2013/0303875, titled “System And Method For Dynamically Varying The Angle Of Light Transmission In An Optoacoustic Imaging System” filed Nov. 02, 2012;

U.S. Pat. No. 9,445,785, titled “System And Method For Normalizing Range In An Optoacoustic Imaging System” filed Dec. 21, 2012;

U.S. Pat. No. 9,282,899, titled “System And Method For Detecting Anomalous Channel In An Optoacoustic Imaging System” filed Dec. 21, 2012;

U.S. Publication 2014/0005544, titled “System And Method For Providing Selective Channel Sensitivity In An Optoacoustic Imaging System” filed Dec. 21, 2012;

U.S. Publication 2016/0317034, titled “System And Method For Providing Selective Channel Sensitivity In An Optoacoustic Imaging System” filed Jul. 11, 2016;

U.S. Pat. No. 9,445,786, titled “Interframe Energy Normalization In An Optoacoustic Imaging System” filed Jan. 22, 2013;

U.S. Publication 2017/0000354, titled “Interframe Energy Normalization In An Optoacoustic Imaging System” filed Sep. 19, 2016;

U.S. Publication 2014/0206978, titled “Probe With Optoacoustic Isolator” filed Jan. 22, 2013;

U.S. Pat. No. 9,743,839, titled “Playback Mode In An Optoacoustic Imaging System” filed Mar. 15, 2013;

U.S. Publication 2017/0332916, titled “Playback Mode In An Optoacoustic Imaging System” filed Jul. 27, 2017;

U.S. Pat. No. 9,398,893, titled “System And Method For Diagnostic Vector Classification Support” filed Mar. 11, 2014;

U.S. Pat. No. 10,026,170, titled “System And Method For Diagnostic Vector Classification Support” filed Jul. 19, 2016

U.S. application Ser. No. 16/022,138, titled “System And Method For Diagnostic Vector Classification Support” filed Jun. 28, 2018;

U.S. Pat. No. 9,730,587, titled “Diagnostic Simulator” filed Mar. 15, 2013;

U.S. Publication 2017/0332915, titled “Diagnostic Simulator” filed Jul. 27, 2017;

U.S. Pat. No. 8,823,928, titled “Light Output Calibration In An Optoacoustic System” filed Mar. 15, 2013;

U.S. Pat. No. 9,163,980, titled “Light Output Calibration In An Optoacoustic System” filed Jul. 11, 2014;

U.S. Pat. No. 9,814,394, titled “Noise Suppression In An Optoacoustic System” filed Mar. 15, 2013;

U.S. Publication 2018/0078144, titled “Noise Suppression In An Optoacoustic System” filed Nov. 13, 2017;

U.S. Pat. No. 9,733,119, titled “Optoacoustic Component Utilization Tracking” filed Mar. 15, 2013;

U.S. Publication 2017/0322071, titled “Optoacoustic Component Utilization Tracking” filed Jul. 27, 2017;

U.S. Publication 2015/0101411, titled “Systems And Methods For Component Separation In Medical Imaging” filed Oct. 13, 2014;

U.S. Publication 2015/0305628, titled “Probe Adapted To Control Blood Flow Through Vessels During Imaging And Method Of Use Of Same” filed Feb. 27, 2015.

U.S. Publication 2016/0187481, titled “Opto-Acoustic Imaging System With Detection Of Relative Orientation Of Light Source And Acoustic Receiver Using Acoustic Waves” filed Oct. 30, 2015.

As used herein, the term “phantom” refers to any and all objects, items, or otherwise utilized to be imaged by an imaging device, typically for practicing using the imaging device. The phantom may replicate, or mimic, any body part, including the breast, prostrate, or the like. The imaging device may include an optoacoustic imaging device, an ultrasound imaging device, MRI device, etc. The phantom may include components, including but not limited to wire elements that replicate irregularities within the replicated body part for imaging.

As used herein, the term “target” refers to any and all components, plural wire elements, plastics, metals, ceramics, nylon wire elements, glass beads or the like that are part of, or within a phantom. The targets represent irregularities that physicians may image when utilizing a medical imaging device in association with a phantom.

As used herein, the term “irregularity” refers to any and all contrasting agents, potential tumors, growths, cluster of cells, or the like detected in an image generated by a medical imaging device based on return signals. The irregularity includes all irregularities including imaged by a medical imaging device, and all false irregularities, artifacts, etc. also detected and imaged by the probe. The irregularities can characterize resolution, sensitivity, specificity, or the like.

As used herein the term “scattering material” refers to any and all materials that include a base material with a reflective material that is suspended within the base material, wherein the reflective material reflects light. In an example, the base material can be mineral oil, while the reflective material can be titanium dioxide (TiO2), a urethane pigment, or the like.

As used herein the term “absorbing material” refers to any and all materials that include a base material with an absorbing material that is suspended with the base material, wherein the absorbing material absorbs light. In an example, the base material can be mineral oil, while the absorbing material can be carbon black with asphaltine.

As used herein the term “tissue material” refers to any and all mixtures, materials, etc. that have optical properties that replicate, mimic, represent, etc. optical properties of tissue of a body part of a human. To replicate, mimic, represent, etc. the optical properties of the tissue, the optical properties of the mixture, materials, etc. are consistent, similar to, or the like to human tissue. The tissue material can include both a scattering material and an absorbing material.

As used herein the term “phantom material” refers to any and all mixtures, materials, etc. that can cure to form a phantom. In one example, the phantom material can be mixtures of the tissue material and a urethane solution.

Provided is a phantom, and method for manufacturing the same that may be utilized to practice imaging utilizing an optoacoustic system. The manufacturing process includes mixing a scattering material with an absorbing material to form a tissue material before adding the tissue material to a urethane solution. The tissue material may then be irradiated with wavelengths of light that will be utilized in practice examination procedures, to ensure the properties of the tissue material are consistent with, or mimic tissue of a human. After the light is absorbed to verify the mimicking of the tissue material, the tissue material is then mixed with the urethane solution and placed in mold for curing. Prior to curing, the mixture of tissue material and urethane solution may with wavelengths of light that will be utilized in practice examination procedures, to ensure the properties of the tissue material are consistent with, or mimic tissue of a human. Within the mold, wire elements, including black, red, green, and clear elements may be arranged to form the phantom. By ensuring the consistency before the addition of the urethane solution, a longer lasting phantom that closely matches the consistency of human tissue is manufactured.

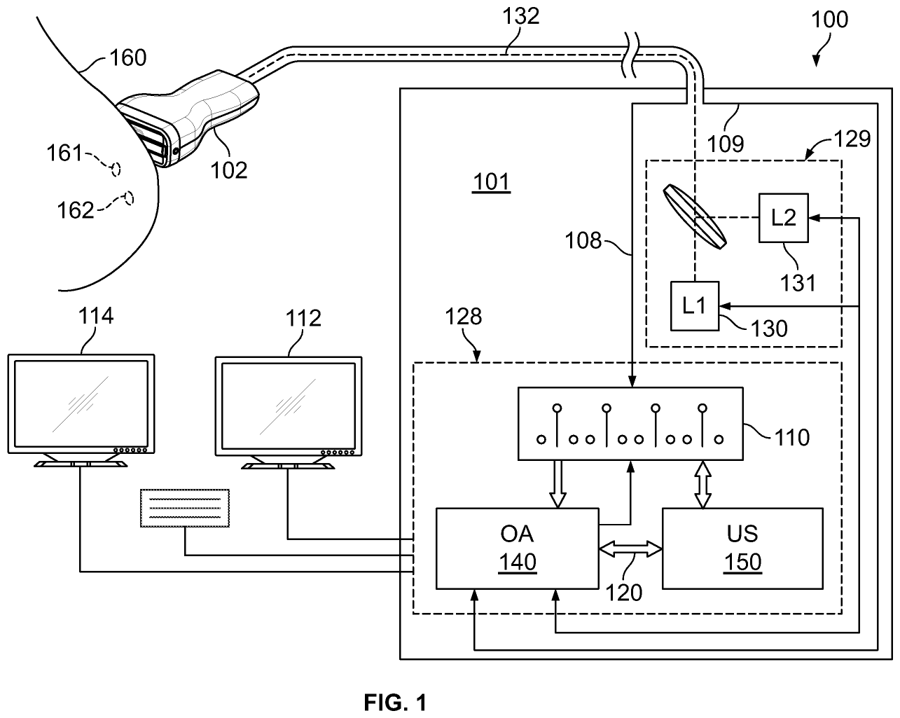

Turning to FIG. 1, generally, device 100 provides an optoacoustic system. In an embodiment, the device 100 includes a probe 102 connected via a light pathway 132 and an electrical path 108 to a system chassis 101. The probe 102 can be utilized to image tissue of a patient in an attempt to locate irregularities 161, 162. In addition, an individual can practice identifying irregularities 161, 162 by utilizing a phantom as described herein. Regarding the optoacoustic system, one or more displays 112, 114, which may be touch screen displays, are provided for displaying images and all or portions of the device 100 user interface. One or more other user input devices (not shown) such as a keyboard, mouse, and various other input devices (e.g., dials and switches) may be provided for receiving input from an operator.

Within the system chassis 101 is housed a light subsystem 129 and a computing subsystem 128. The computing subsystem 128 includes one or more computing components for optoacoustic control and analysis; these components may be separate, or integrated. In an embodiment, the computing subsystem comprises a relay system 110, a triggering system, an optoacoustic processing and overlay system 140 and an ultrasound instrument 150. In one embodiment, the triggering system is configured to actuate and control operation of the light sources 130, 131.

The light sources 130, 131 in one example are lasers that each emit a determined wavelength of light. The light sources 130, 131 are configured to generate the light in order to form the light pathway 132. The light sources 130, 131 are consequently utilized in generating return signals for imaging purposes by generating the light for the light pathway 132 that is located at a scanning area of a volume 160.

The volume 160 can include organic tissue, phantom, or other volume 160 that may have one or more irregularities, or inhomogeneities, 161, 162, such as e.g., a tumor, within. An ultrasound gel (not shown) or other material may be used to improve acoustic coupling between the probe 102 and the surface of the volume 160 and/or to improve optical energy transfer. In use, the probe 102 may be moved by a physician against the volume to locate and identify the irregularities 161, 162.

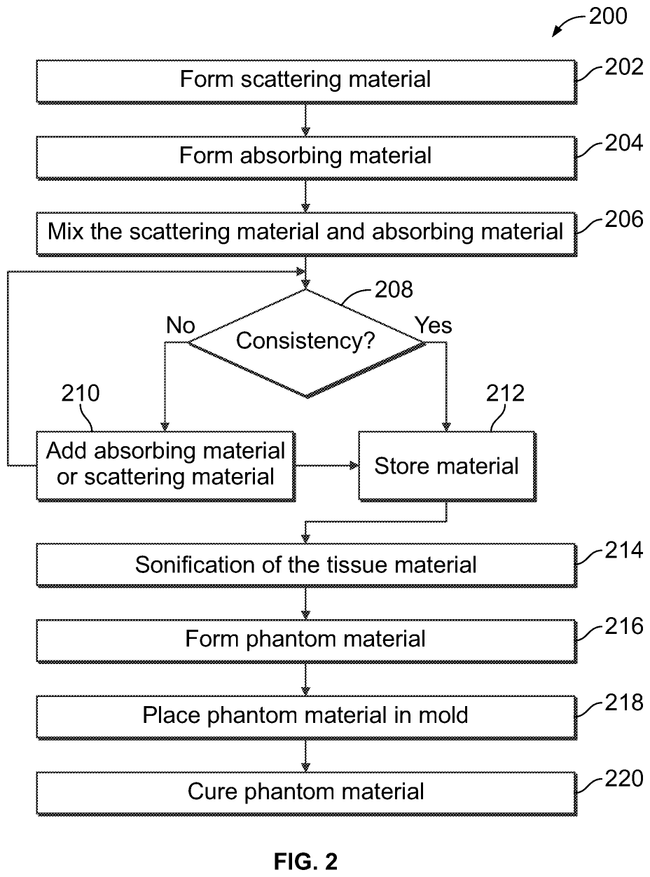

Turning now to FIG. 2, FIG. 2 illustrates a process 200 for manufacturing a phantom that can be utilized with an optoacoustic system to train an individual for imaging with the optoacoustic system. In one example, the optoacoustic system, device, and probe of FIG. 1 are the optoacoustic system, device, and probe as described in the process 200.

At 202, a scattering material is formed. The scattering material includes any and all material that causes light from a light source to reflect or scatter that is not an irregularity. In particular, human tissue include cells, fibers, etc. that result in the scattering, reflection, deflection, etc. of light resulting in a sound wave being produced. Such scattering material must be accounted for when imaging utilizing the optoacoustic system. In the phantom, the scattering material, and in particular, the consistency of the scattering material within the phantom must replicate the consistency of scattering material of a patient.

In one example, the scattering material may be formed utilizing titanium dioxide (TiO2), and a urethane pigment. Because the phantom is to be made of a urethane material that extends the life of the phantom, a urethane pigment is utilized in the scattering material. In another example, a colloidally stable TiO2, and colloidally stable urethane pigment are utilized. As used herein, colloidally stable refers to, and indicates that the TiO2 and urethane pigment are stable in a mixture in which the TiO2 and urethane pigment are dispersed in soluble particles suspended throughout another scattering material substance. In one example, the other scattering material substance is a base material in which the TiO2 is suspended. In an example, the base material is a mineral oil. In one example, the TiO2 and urethane pigment are both microscopic and suspended within the mineral oil to provide the scattering properties of the phantom.

At 204, an absorbing material is formed. The absorbing material includes any and all materials that absorb light within the phantom. In particular, during optoacoustic imaging, certain tissues, fibers, fluids, blood vessels, or the like absorb light emitted by the optoacoustic imaging system. As a result, when imaging with the optoacoustic imaging system, the absorbing material must be accounted for during imaging. In one example, the absorbing material may include carbon black with asphaltine. The carbon black with asphaltine absorbs light to replicate body and tissue components that similarly absorb light. In an example, the carbon black with asphaltine is a colloidally stable carbon black with asphaltine material. Again, the carbon black in asphaltine may be provided as material suspended in a base material, including as microscopic materials suspended in another material. In one example, the base material is mineral oil. In a different example, the base material is the same material that the TiO2 and urethane pigment are suspended in.

At 206, the scattering material and the absorbing material are mixed to form a tissue material. The tissue material provides a consistency of absorbing and scattering materials that would be within the tissue of a typical body part to be imaged by an optoacoustic system. The determination on the amount may be based on historical data of previous patients and the consistency of absorbing material and scattering material found in the tissue of body parts that have been previously scanned.

At 208, a determination is made regarding whether the consistency of the tissue material is consistent with a human tissue. To ensure the phantom replicates the human tissue, the light having a wavelength in a range that will be utilized by an optoacoustic probe is emitted into the tissue material. In one example, the wavelength may be 757 nm such that range of wavelengths is between 740 nm and 770 nm. In another example, the wavelength can be 1064 nm such that the range of wavelengths is between 1050 nm-1070 nm. In another example, both the 757 nm and 1064 nm wavelength utilized to determine whether the consistency of the tissue material is consistent with human tissue. In yet other example, other wavelengths and accompanying ranges of wavelengths are utilized to determine whether the consistency of the tissue material is consistent with human tissue.

If at 208, the consistency of the tissue material is not consistent with human tissue, then at 210, either additional absorbing material and/or scattering material is mixed into the tissue material. At this time, the tissue material can be retested to ensure the consistency is accurate. In this manner, the tissue material may be analyzed before forming the phantom to ensure the phantom mimics human tissue prior to forming the phantom.

If at 208, the consistency of the tissue material is consistent with human tissue, then at 212 the tissue material is stored. In one example, the tissue material is stored in a borosilicate glass container to prevent electrochemical changes to the tissue material.

At 214, prior to making the final phantom, sonification of the tissue material occurs. In particular, any aggregation, clumping, or consistency changes that occur during storage may be mitigated by providing ultrasonic waves to reestablish the spacing and consistency of the materials suspended in the base material.

At 216, the tissue material is mixed with a urethane solution to form a phantom material. Any aggregation, clumping, or the like that may occur during the mixing process may be mitigated by selecting mixing vessels and grounding metal components to minimize electrostatic buildup. At this time, tissue material can be retested to ensure the consistency is accurate. In this manner, the tissue material may be analyzed before curing the phantom to ensure the phantom mimics human tissue. By utilizing a urethane solution, the life of the phantom is increased compared to other phantoms that do not utilize a urethane solution.

At 218, the phantom material is placed into a mold. When in the mold, one or more targets may be placed in the phantom material. Targets are materials that represent irregularities within the phantom that should be able to be identified by a physician that is imaging utilizing an optoacoustic probe. The targets may be arranged with specific patterns, spaced, or the like to assist in training the physician. In one example the targets may be plural wire elements, including nylon wire elements. In another example, the wire elements may vary in color. In an example, the wire elements may include black wire elements, red wire elements, green wire elements, and clear wire elements. In one example wires are provided that are responsive to the different wavelengths of the system (Alexandrite laser at 757 nm and Nd:YAG laser at 1064 nm), which are visualized as red and green. Thus, while the wires are not physically red or green in color, they are different materials with different absorption properties to produce different OA responses to appear red and green when viewed. By providing the red wire elements and the green wire elements in addition to the black and clear wire elements, enhanced training can be provided.

At 220, the phantom material is cured to form the phantom. Once the targets are arranged, the phantom material is cured in order to form the phantom that include plural targets and utilized by a physician for training.

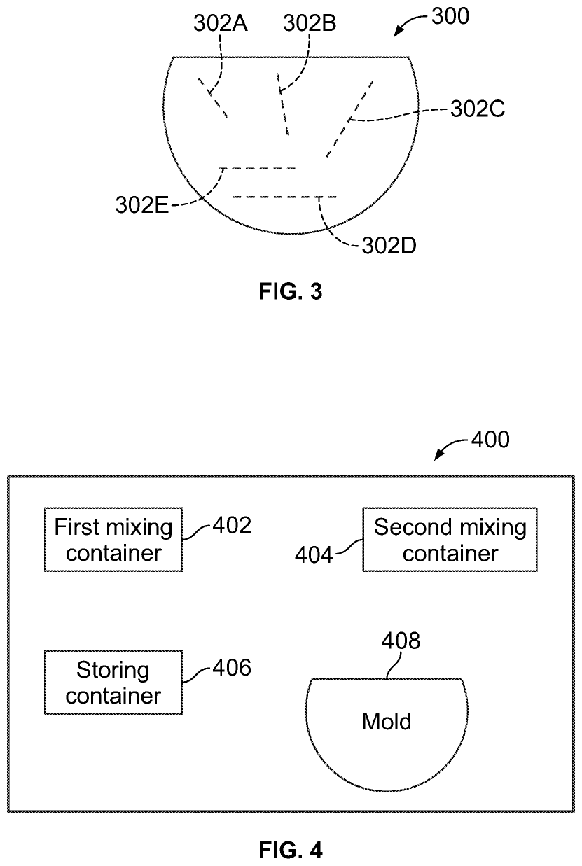

Turning now to FIG. 3, illustrates an example phantom 300 manufactured utilizing the method of FIG. 2. In one example, the phantom 300 can be utilized in association with a device, probe, or optoacoustic system as described in relation to FIG.1 so that an individual can practice utilizing the probe to identify targets 302A-302E that are representative of irregularities. In one example, the targets may include plural wire elements. The plural wire elements can be black wire elements, red wire elements, green wire elements, and clear wire elements.

Turning to FIG. 4, illustrates a manufacturing system 400 that may be utilized for forming a phantom. In one example, the phantom is the phantom of FIG. 3. In another example, the manufacturing system 400 may be utilized to perform one or more steps of the process described in relation to FIG. 2.

The system may include a first mixing container 402 for receiving scattering material and absorbing material. In one example, the scattering material includes TiO2 and a base material that can be mineral oil. Meanwhile the absorbing material can include carbon black with asphaltine and a base material, that is the same base material as the scattering material. In one example the base material is mineral oil.

In addition to the first mixing container 402 the system 400 can include a storing container 406 that in one example is a borosilicate glass container. The storing container 406 in example embodiments can be sealed, including under pressure.

The system 400 can also include a mold 408 that receives the phantom material. In example embodiments the tissue material can be mixed with the urethane solution within the mold, or in a second mixing container 404. The mold 408 can be configured to receive plural wire elements. The mold 408 can also be configured to allowing curing of the phantom material at room temperature (e.g. 65° F. to 75° F.) or at an elevated temperature (e.g. 200° F. or greater). Once cured, the mold 408 and accompanying phantom may be cooled to room temperature (when curing occurs at an elevated temperature) to allow the phantom to be removed.

The present system and methods are described above with reference to block diagrams and operational illustrations of methods and devices comprising an optoacoustic probe.

It is understood that each block of the block diagrams or operational illustrations, and combinations of blocks in the block diagrams or operational illustrations, may be implemented by means of analog or digital hardware and computer program instructions. These computer program instructions may be provided to a processor of a general purpose computer, special purpose computer, ASIC, FPGA, or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g. 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about,” unless the context clearly dictates otherwise. Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Those skilled in the art will recognize that the methods and systems of the present disclosure may be implemented in many manners and as such are not to be limited by the foregoing example embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces, and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto. Thus, while the invention has been particularly shown and described with reference to a preferred embodiment thereof, it will be understood by those skilled in the art that various changes in form and details may be made therein without departing from the spirit and scope of the invention.

What is claimed is:

1. A method for forming a phantom for an optoacoustic probe comprising:
forming a scattering material;
forming an absorbing material;
mixing the scattering material with the absorbing material to form a tissue material mixing the tissue material with a urethane solution to form a phantom material; and
curing the phantom material to form the phantom.

2. The method of claim 1, wherein the scattering material include TiO2 and a base material.

3. The method of claim 2, wherein the base material is mineral oil.

4. The method of claim 1, wherein the absorbing material includes carbon black with asphaltine and a base material.

5. The method of claim 1, further comprising determining whether the tissue material absorbs light related to a range of wavelengths.

6. The method of claim 5, wherein the range of wavelengths is at least one of between 740 nm and 770 nm, or 1050 nm-1070 nm.

7. The method of claim 5, wherein either one of additional scattering material or additional absorbing material is mixed with the tissue material based on determining whether the tissue material absorbs light related to the range of wavelengths.

8. The method of claim 1, wherein the curing of the phantom material to form the phantom occurs at a temperature in a range of between 65° -75° F.

9. The method of claim 1, wherein prior to mixing the tissue material with the urethane solution to form the phantom material includes sonification of tissue material.

10. The method of claim 1, further comprising, placing the phantom material in a mold; and
arranging one or more targets within the phantom material within the mold.

11. The method of claim 10, wherein the one or more targets include plural wire elements.

12. The method of claim 10, wherein the plural wire elements include a black wire element, red wire element, green wire element, and a clear wire element.

13. A method for forming a phantom for an optoacoustic probe comprising:
forming a scattering material that includes TiO2 and a base material;
forming an absorbing material that includes carbon black with asphaltine and a base material;
mixing the scattering material with the absorbing material to form a tissue material;
mixing the tissue material with a urethane solution to form a phantom material; and
curing the phantom material to form the phantom.

14. The method of claim 13, wherein the base material is mineral oil.

15. The method of claim 13, further comprising determining whether the tissue material absorbs light related to a range of wavelengths including at least one of between 740 nm and 770 nm, or 1050 nm-1070 nm.

16. The method of claim 13, wherein the curing of the phantom material to form the phantom occurs at a temperature in a range of between 65° -75° F.

17. The method of claim 13, further comprising, placing the phantom material in a mold; and
arranging one or more targets within the phantom material within the mold.

18. A method for forming a phantom for an optoacoustic probe comprising:
forming a scattering material that includes TiO2 and mineral oil;
forming an absorbing material that includes carbon black with asphaltine and mineral oil;
mixing the scattering material with the absorbing material to form a tissue material;
storing the tissue material;
performing sonification of the tissue material;
mixing the tissue material with a urethane solution to form a phantom material; and
curing the phantom material to form the phantom.

19. The method of claim 18, further comprising determining whether the tissue material absorbs light related to a range of wavelengths.

20. The method of claim 19, wherein the range of wavelengths is at least one of between 740 nm and 770 nm, or 1050 nm-1070 nm.

21. The method of claim 1, wherein the scattering material includes a base material with a reflective material that is suspended within the base material.

22. The method of claim 1, wherein the absorbing material includes a base material with an absorbing material that is suspended with the base material.

23. The method of claim 1, wherein the tissue material has optical properties that replicate optical properties of tissue of a body part.
